(19) United States
(12) Patent Application Publication
(10) Pub. No.: US 2016/0143923 A1
(43) Pub. Date: May 26, 2016

(54) LOW DOSE PHARMACEUTICAL COMPOSITION OF DOXYCYCLINE

(71) Applicant: RANBAXY LABORATORIES LIMITED, New Delhi (IN)

(72) Inventors: Anuj Kumar FANDA, Ghaziabad (IN); Romi Barat SINGH, Varanasi (IN)

(21) Appl. No.: 14/548,915

(22) Filed: Nov. 20, 2014

(51) Int. Cl. A61K 31/65

(52) U.S. Cl. CPC A61K 31/65

(57) ABSTRACT

The present invention relates to a low dose pharmaceutical composition comprising 32 mg or 34 mg doxycycline and one or more pharmaceutically acceptable excipients for the treatment of acne, and processes for its preparation.

### FIELD OF THE INVENTION

The present invention relates to a low dose pharmaceutical composition of doxycycline comprising 32 mg or 34 mg of doxycycline and one or more pharmaceutically acceptable excipients for the treatment of acne, and processes for its preparation.

### BACKGROUND OF THE INVENTION

U.S. Pat. No. 8,652,516 discloses a pharmaceutical composition of doxycycline comprising a capsule, wherein the capsule is coated with a delayed-release layer which comprises about 4 mg to 6 mg of doxycycline and an immediate-release layer which comprises about 32 mg of doxycycline. The prior art discloses 36 mg and 38 mg doxycycline capsule formulations which are bioequivalent to the reference product Oracea® 40 mg.

Doxycycline has gastrointestinal side effects which are dose related such as gastrointestinal irritation. Thus lowering the dose of doxycycline may reduce these side effects. The present inventors have prepared a pharmaceutical composition which further lowers the dose of doxycycline for the treatment of acne.

The present invention is directed to a low dose pharmaceutical composition comprising 32 mg or 34 mg doxycycline which is effective in the treatment of acne.

### SUMMARY OF THE INVENTION

The present invention provides a low dose pharmaceutical composition of doxycycline comprising 32 mg or 34 mg of doxycycline and one or more pharmaceutically acceptable excipients for the treatment of acne, and processes for its preparation.

### DETAILED DESCRIPTION OF THE INVENTION

A first aspect of the present invention provides a low dose pharmaceutical composition comprising 32 mg or 34 mg of doxycycline and one or more pharmaceutically acceptable excipients.

According to one embodiment of this aspect, the low dose pharmaceutical composition is intended for once daily administration.

According to another embodiment of this aspect, the low dose pharmaceutical composition is an immediate release composition.

A second aspect of the present invention provides a method of treating acne by administering a low dose pharmaceutical composition comprising 32 mg or 34 mg of doxycycline and one or more pharmaceutically acceptable excipients.

The pharmaceutical composition may be in the form of capsules or tablets. Preferably, the pharmaceutical composition is in the form of tablets.

The term “doxycycline,” as used herein, includes doxycycline base and its pharmaceutically acceptable salts, hydrates, solvates, esters, or prodrugs. Preferably, doxycycline is used as its hyclate salt, which is doxycycline hydrochloride hemiethanolate hemihydrate.

The term “low dose,” as used herein, refers to a dose of 32 mg or 34 mg doxycycline base, which is less than the conventional dose of 40 mg which is required to produce the therapeutic effect.

The term “pharmaceutically acceptable excipients,” as used herein, includes any physiologically inert additives that are routinely used in pharmaceutical compositions. Pharmaceutically acceptable excipients are selected from the group comprising binders, diluents, disintegrants, lubricants/glidants/antiadherants, and mixtures thereof. The pharmaceutically acceptable excipients may be added intragranularly as well as extragranularly.

Examples of binders include povidone, copovidone, polyvinylpyrrolidone, hydroxypropylmethyl cellulose, hydroxypropyl cellulose, hydroxyethyl cellulose, methyl cellulose, ethyl cellulose, xanthan gum, gum acacia, gum arabic, tragacanth, sorbitol, dextrose, sucrose, mannitol, gelatin, pullulan, sodium alginate, calcium alginate, ammonium calcium alginate, propylene glycol, polyvinyl alcohol, corn syrup, methacrylates, carboxyvinyl polymers like carbomers, and mixtures thereof.

Examples of diluents include microcrystalline cellulose, powdered cellulose, dibasic calcium phosphate, tribasic calcium phosphate, calcium sulfate, calcium carbonate, lactose monohydrate, lactose anhydrous, sucrose, sorbitol, xylitol, erythritol, kaolin, calcium silicate, maltodextrin, starch, modified starch, e.g., pregelatinized starch, maize starch, corn starch, and mixtures thereof.

Examples of disintegrants include hydroxypropyl cellulose (L-HPC), crospovidone, croscarmellose sodium, carboxymethyl cellulose sodium, carboxymethyl cellulose calcium, sodium starch glycolate, gums, alginic acid or alginates, starch, corn starch, modified starch, carboxymethyl starch, polyacrylates, and mixtures thereof.

Examples of lubricants/glidants/antiadherents include magnesium stearate, hydrogenated vegetable oil, glyceryl behenate, glyceryl monostearate, stearic acid, sodium stearyl fumarate, calcium stearate, zinc stearate, aluminum silicate, talc, colloidal silicon dioxide, sucrose esters of fatty acids, waxes, silica gel, and mixtures thereof.

Various solvents that may be employed during the preparation of the pharmaceutical composition of the present invention are selected from the group comprising methyl alcohol, ethyl alcohol, isopropyl alcohol, n-butyl alcohol, acetone, acetonitrile, chloroform, methylene chloride, water, and mixtures thereof.

The pharmaceutical composition of the present invention may be prepared by any of the well-known processes including wet granulation, dry granulation, direct compression, top spray granulation and drug layering.

The pharmaceutical composition of the present invention may be in the form of a tablet or a capsule. The tablet or capsule may be further coated with a film coating prepared by using a film-forming polymer and one or more pharmaceutically acceptable excipients. The pharmaceutically acceptable excipients may be plasticizers, opacifiers, coloring agents, and mixtures thereof.

Examples of film-forming polymers include hydroxypropylmethyl cellulose, ethyl cellulose, methyl cellulose, hydroxyethyl cellulose, hydroxypropyl cellulose, sodium carboxymethyl cellulose, cellulose acetate, hydroxypropylmethyl cellulose phthalate, cellulose acetate trimellitate, methacrylic acid copolymers, e.g., Eudragit®, polyvinylpyrrolidone, polyvinylalcohol, polyethylene glycol, and mixtures thereof. A preferred film-forming polymer is hydroxypropylmethyl cellulose. Other suitable film-forming polymers which are known in the art may also be used. Many suitable film coating products which are commercially available, e.g., Opadry® and Opaglos®, may be used.

Examples of plasticizers include propylene glycol, triethyl citrate, tributyl citrate, dibutyl sebacate, acetyl tributyl citrate, glyceryl monostearate, triacetin, polyethylene glycol, diethyl phthalate, acetylated monoglycerides, diacetylated monoglycerides, cetyl alcohol, and mixtures thereof.

Examples of opacifiers include titanium dioxide, manganese dioxide, iron oxide, silicon dioxide, and mixtures thereof.

The coloring agents may be selected from FDA approved colorants such as iron oxide, lake of tartrazine, allura red, titanium dioxide, and mixtures thereof.

The coating may be carried out by using any conventional coating techniques known in the art, such as spray coating in a conventional coating pan or fluidized bed processor, or dip coating.

The following examples illustrate the present invention but are not to be construed as limiting the scope of the invention.

### EXAMPLES

### Example 1

| | Quantity |
|---|---|
| Ingredients | (% w/w) |
| Doxycycline hyclate equivalent to doxycycline 32 mg | 18.47 |
| Hydroxypropylmethyl cellulose | 1.54 |
| Sugar spheres | 80.00 |
| Purified water | q.s. |

### Manufacturing Process:

1. Hydroxypropylmethyl cellulose is dissolved in purified water to form a binder solution.
2. Doxycycline is added to the binder solution of step 1 to form a drug-binder solution.
3. Sugar spheres are coated with the drug-binder solution of step 2 to obtain coated pellets.
4. The coated pellets of step 3 are dried, then filled into capsules.

### Example 2

| | Quantity |
|---|---|
| Ingredients | (% w/w) |
| Doxycycline hyclate equivalent to doxycycline 34 mg | 19.62 |
| Hydroxypropylmethyl cellulose | 2.38 |
| Sugar spheres | 78.00 |
| Purified water | q.s. |

### Manufacturing Process:

1. Hydroxypropylmethyl cellulose is dissolved in purified water to form a binder solution.
2. Doxycycline is added to the binder solution of step 1 to form a drug-binder solution.
3. Sugar spheres are coated with the drug-binder solution of step 2 to obtain coated pellets.
4. The coated pellets of step 3 are dried, then filled into capsules.

### Example 3

| | Quantity |
|---|---|
| Ingredients | (% w/w) |
| Doxycycline hyclate equivalent to doxycycline 32 mg | 18.47 |
| Polyvinyl pyrrolidone | 3.04 |
| Microcrystalline cellulose | 77.50 |
| Purified water | q.s. |
| Magnesium stearate | 1.00 |

### Manufacturing Process:

1. Polyvinyl pyrrolidone is dissolved in purified water to form a binder solution.
2. Doxycycline and microcrystalline cellulose are mixed together to obtain a blend.
3. The blend of step 2 is granulated using the binder solution of step 1.
4. The granules of step 3 are lubricated with magnesium stearate.
5. The lubricated granules of step 4 are compressed into tablets.

### Example 4

| | Quantity |
|---|---|
| Ingredients | (% w/w) |
| Doxycycline hyclate equivalent to doxycycline 34 mg | 19.62 |
| Polyvinyl pyrrolidone | 2.38 |
| Microcrystalline cellulose | 77.00 |
| Purified water | q.s. |
| Magnesium stearate | 1.00 |

### Manufacturing Process:

1. Polyvinyl pyrrolidone is dissolved in purified water to form a binder solution.
2. Doxycycline and microcrystalline cellulose are mixed together to obtain a blend.
3. The blend of step 2 is granulated using the binder solution of step 1.
4. The granules of step 3 are lubricated with magnesium stearate.
5. The lubricated granules of step 4 are compressed into tablets.

### Example 5

| | Quantity |
|---|---|
| Ingredients | (% w/w) |
| Doxycycline hyclate equivalent to doxycycline 32 mg | 18.47 |
| Polyvinyl pyrrolidone | 3.04 |
| Microcrystalline cellulose | 77.50 |
| Purified water | q.s. |
| Magnesium stearate | 1.00 |

### Manufacturing Process:

1. Polyvinyl pyrrolidone is dissolved in purified water to form a binder solution.
2. Doxcycline is added to the binder solution of step 1 to form a drug-binder solution.
3. Microcrystalline cellulose is granulated using the drug-binder solution of step 2.
4. The granules of step 3 are lubricated with magnesium stearate.
5. The lubricated granules of step 4 are compressed into tablets.

### Example 6

| | Quantity |
|---|---|
| Ingredients | (% w/w) |
| Doxycycline hyclate equivalent to doxycycline 34 mg | 19.62 |
| Polyvinyl pyrrolidone | 2.38 |
| Microcrystalline cellulose | 77.00 |
| Purified water | q.s. |
| Magnesium stearate | 1.00 |

### Manufacturing Process:

1. Polyvinyl pyrrolidone is dissolved in purified water to form a binder solution.
2. Doxcycline is added to the binder solution of step 1 to form a drug-binder solution.
3. Microcrystalline cellulose is granulated using the drug-binder solution of step 2.
4. The granules of step 3 are lubricated with magnesium stearate.
5. The lubricated granules of step 4 are compressed into tablets.

### Example 7

| | Quantity |
|---|---|
| Ingredients | (% w/w) |
| Doxycycline hyclate equivalent to doxycycline 32 mg | 18.47 |
| Polyvinyl pyrrolidone | 3.54 |
| Microcrystalline cellulose | 77.00 |
| Magnesium stearate | 1.00 |

### Manufacturing Process:

1. Doxycycline, microcrystalline cellulose, and polyvinyl pyrrolidone are mixed together to obtain a blend.
2. The blend of step 1 is lubricated with magnesium stearate and compressed into tablets.

### Example 8

| | Quantity |
|---|---|
| Ingredients | (% w/w) |
| Doxycycline hyclate equivalent to doxycycline 34 mg | 19.62 |
| Polyvinyl pyrrolidone | 2.38 |
| Microcrystalline cellulose | 77.00 |
| Magnesium stearate | 1.00 |

### Manufacturing Process:

1. Doxycycline, microcrystalline cellulose and polyvinyl pyrrolidone are mixed together to obtain a blend.
2. The blend of step 1 is lubricated with magnesium stearate and compressed into tablets.

### Example 9

| | Quantity |
|---|---|
| Ingredients | (% w/w) |
| Doxycycline monohydrate equivalent to doxycycline 32 mg | 17.69 |
| Hydroxypropylmethyl cellulose | 2.91 |
| Sugar spheres | 79.40 |
| Purified water | q.s. |

### Manufacturing Process:

1. Hydroxypropylmethyl cellulose is dissolved in purified water to obtain a binder solution.
2. Doxycycline is added to the binder solution of step 1 to form a drug-binder solution.
3. Sugar spheres are coated with the drug-binder solution of step 2 to obtain coated pellets.
4. The coated pellets of step 3 are dried, then filled into capsules.

### Example 10

| | Quantity |
|---|---|
| Ingredients | (% w/w) |
| Doxycycline monohydrate equivalent to doxycycline 34 mg | 17.69 |
| Polyvinyl pyrrolidone | 2.48 |
| Microcrystalline cellulose | 78.83 |
| Magnesium stearate | 1.00 |

### Manufacturing Process:

1. Doxycycline, microcrystalline cellulose, and polyvinyl pyrrolidone are mixed together to obtain a blend.
2. The blend of step 1 is lubricated with magnesium stearate and compressed into tablets.

### Example 11

| | Quantity |
|---|---|
| Ingredients | (% w/w) |
| Doxycycline monohydrate equivalent to doxycycline 32 mg | 16.65 |
| Polyvinyl pyrrolidone | 3.86 |
| Microcrystalline cellulose | 78.50 |
| Purified water | q.s. |
| Magnesium stearate | 1.00 |

### Manufacturing Process:

1. Polyvinyl pyrrolidone is dissolved in purified water to form a binder solution.
2. Doxcycline is added to the binder solution of step 1 to form a drug-binder solution.
3. Microcrystalline cellulose is granulated using the drug-binder solution of step 2.
4. The granules of step 3 are lubricated with magnesium stearate.
5. The lubricated granules of step 4 are compressed into tablets.

1-4. (canceled)

5. A low dose capsule or tablet comprising a pharmaceutical composition that comprises 32 mg or 34 mg of doxycycline and one or more pharmaceutically acceptable excipients, wherein said capsule or tablet comprise a total of 32 mg or 34 mg of doxycycline.

6. The low dose capsule or tablet of claim 5, wherein said pharmaceutical composition is an immediate release composition.

7. The low dose capsule or tablet of claim 5, wherein said pharmaceutical composition is intended for once daily administration.

8. The low dose capsule or tablet of claim 5, wherein the pharmaceutically acceptable excipients are selected from the group comprising binders, diluents, disintegrants, lubricants/glidants/antiadherants, and mixtures thereof.
